(19) United States
(12) Patent Application Publication
(10) Pub. No.: US 2026/0224159 A1
(43) Pub. Date: Aug. 6, 2026

(54) OPTICAL DUAL BRAIN SIGNAL MEASUREMENT DEVICE

(71) Applicant: DAEGU GYEONGBUK INSTITUTE OF SCIENCE AND TECHNOLOGY, Daegu (KR)

(72) Inventors: Kwang LEE, Daegu (KR); Young Kwon PARK, Bucheon-si, Gyeonggi-do (KR); Hyo Won KIM, Daegu (KR); Min Yong LEE, Sejong (KR)

(21) Appl. No.: 19/150,466

(22) PCT Filed: Jun. 12, 2024
(86) PCT No.: PCT/KR2024/008015

§ 371 Date: Jul. 23, 2025

(30) Foreign Application Priority Data
Jul. 5, 2023 (KR) 10-2023-0087177

(51) Int. Cl. A61B 5/00

(52) U.S. Cl. CPC A61B 5/4058; A61B 5/0017; A61B 5/0082; A61B 2562/223

(57) ABSTRACT

An optical brain signal measurement device according to one embodiment may comprise: a light source unit; an excitation filter unit; a first segmentation/concatenation unit; a second segmentation/concatenation unit; an emission filter unit; and a measurement unit. The optical brain signal measurement device can segment or concatenate optical signals through a segmentation/concatenation unit while transmitting the signals to a brain signal measurement target through an optical fiber having directivity, and identify the wavelength range to which each of multiple signals correspond through a spectrometer to obtain multiple brain signals (multiple light sources) by using the light source directivity of the optical fiber and the spectrometer without a dichroic filter.

### TECHNICAL FIELD

The present invention relates to an optical brain signal measurement device and a method thereof, and more particularly, to an optical fiber photometry technique for acquiring multiple brain signals by using a light source directionality and a light source classification function of an optical fiber and a spectrometer without a dichroic filter.

### BACKGROUND ART

The optical fiber photometry technique uses a device that emits light to a target sample using an optical fiber and receives the light back through the optical fiber.

The optical fiber photometry technique is applied to neural activity researches as it can measure optical signals generated at a position deep in the brain of a living animal.

Among the methods of measuring brain signals in a non-invasive manner, there is a technique that extracts and amplifies optical brain response signals using a lock-in amplifier.

In the method of extracting and amplifying optical brain response signals using a lock-in amplifier, a photodetector is used.

There are techniques that use a method of measuring brain signals by removing auto-fluorescence and noise by adding a 405 nm light source, a method of transferring brain signals of multiple regions (multiple objects) to a CMOS camera, rather than a photodetector, using a multimode fiber and measuring the brain signals as a regional light source value using software, or the like.

Since the brain signals are defined as a value obtained by optically calculating the intensity of fluorescence recorded by the CMOS camera, not an actually measured light source value, and the calculated value is stored as a brain signal, there is a time difference between the measured value and the calculated value.

The fiber photometry, including the method of measuring brain signals in a noninvasive manner, which optically records brain signals, necessarily requires an excitation filter, a dichroic filter, and an emission filter, since filters for setting the directionality and wavelength range of light are required.

According to the conventional techniques, a dichroic filter is additionally required to record multiple regions and multiple objects using multiple optical fibers, and lenses, dichroic filters, and photodetectors are additionally required to record multiple brain signals according to addition of light sources.

### DISCLOSURE OF INVENTION

### Technical Problem

Therefore, the present invention has been made in view of the above problems, and it is an object of the present invention is to acquire multiple brain signals by using a light source directionality and a light source classification function of an optical fiber and a spectrometer without a dichroic filter.

Another object of the present invention is to provide an optical fiber photometry technique for acquiring multiple brain signals by utilizing an optical fiber having a specific directionality and a filter function without various lenses and filters.

Another object of the present invention is to provide an optical brain signal measurement device and method capable of simultaneous recording of light sources of multiple brain signals without configuration of various filters although intuitive and monotonous.

### Technical Solution

To accomplish the above objects, according to one aspect of the present invention, there is provided an optical brain signal measurement device comprising: a light source unit for inputting an optical signal of a first wavelength and an optical signal of a second wavelength; an excitation filter unit for transmitting the optical signal of a first wavelength in a first wavelength range, and transmitting the optical signal of a second wavelength in a second wavelength range; a first segmentation/concatenation unit for receiving the optical signal transmitted in a first wavelength range and the optical signal transmitted in a second wavelength range on the basis of a first directionality of an optical fiber, and outputting a concatenation signal concatenating the transferred optical signals or segmentation signals segmenting a concatenation signal; a second segmentation/concatenation unit for transferring any one signal among the concatenation signal and the segmentation signal to at least one brain signal measurement target on the basis of the first directionality of the optical fiber, and transferring a measurement signal returning from the at least one brain signal measurement target on the basis of a second directionality of the optical fiber; an emission filter unit for transmitting the transferred measurement signal in at least one wavelength range among a third wavelength range and a fourth wavelength range; and a measurement unit for measuring at least one brain signal of the at least one brain signal measurement target on the basis of the measurement signal transmitted in at least one wavelength range.

The first directionality of the optical fiber may represent a directionality of transferring an optical signal from the light source unit to the excitation filter unit, transferring the optical signal from the excitation filter unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality of the optical fiber may represent a directionality of transferring an optical signal returning from the at least one brain signal measurement target to the second segmentation/concatenation unit, transferring the optical signal from the second segmentation/concatenation unit to the emission filter unit, and transferring the optical signal from the emission filter unit to the measurement unit.

When the at least one brain signal measurement target is a plurality of brain signal measurement targets, the second segmentation/concatenation unit, the emission filter unit, and the measurement unit may be additionally configured as many as the number of the plurality of brain signal measurement targets.

The first segmentation/concatenation unit may output a concatenation signal concatenating the transferred optical signals when the at least one brain signal measurement target is one brain signal measurement target, and output segmentation signals segmenting a concatenation signal when the at least one brain signal measurement target is a plurality of brain signal measurement targets.

In relation to the segmentation signals, the first segmentation/concatenation unit may adjust a segmentation ratio to any one segmentation ratio among 1:10 to 10:1.

The measurement unit may be a spectrometer when the measurement signal transmitted in at least one wavelength range is in a plurality of wavelength ranges, and a photodetector when the measurement signal transmitted in at least one wavelength range is in one wavelength range.

According to another aspect of the present invention, there is provided an optical brain signal measurement device comprising: a light source unit for transferring an optical signal of a first wavelength and an optical signal of a second wavelength through a first optical fiber that transfers the optical signal of a first wavelength transmitted in a first wavelength range and the optical signal of a second wavelength transmitted in a second wavelength range in the first directionality; a first segmentation/concatenation unit for outputting a concatenation signal concatenating the optical signal transmitted in a first wavelength range and the optical signal transmitted in a second wavelength range or segmentation signals segmenting a concatenation signal through the first optical fiber; a second segmentation/concatenation unit for transferring any one signal among the concatenation signal and the segmentation signal to at least one brain signal measurement target through the first optical fiber, and transferring a measurement signal returning from the at least one brain signal measurement target through a second optical fiber that transmits the measurement signal in at least one wavelength range among a third wavelength range and a fourth wavelength range and transfers in the second directionality; and a measurement unit for measuring at least one brain signal of the at least one brain signal measurement target on the basis of the transferred measurement signal.

The first directionality may represent a directionality of transferring an optical signal from the light source unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality may represent a directionality of transferring an optical signal returning from the at least one brain signal measurement target to the second segmentation/concatenation unit, and transferring the optical signal from the second segmentation/concatenation unit to the measurement unit.

When the at least one brain signal measurement target is a plurality of brain signal measurement targets, the second segmentation/concatenation unit and the measurement unit may be additionally configured as many as the number of the plurality of brain signal measurement targets.

The first segmentation/concatenation unit may output a concatenation signal concatenating the transferred optical signals when the at least one brain signal measurement target is one brain signal measurement target, and output segmentation signals segmenting a concatenation signal when the at least one brain signal measurement target is a plurality of brain signal measurement targets.

In relation to the segmentation signals, the first segmentation/concatenation unit may adjust a segmentation ratio to any one segmentation ratio among 1:10 to 10:1.

The measurement unit may be at least one among a spectrometer and a photodetector.

According to another aspect of the present invention, there is provided an optical brain signal measurement method comprising the steps of: inputting an optical signal of a first wavelength and an optical signal of a second wavelength, by a light source unit; transmitting the optical signal of a first wavelength in a first wavelength range, and transmitting the optical signal of a second wavelength in a second wavelength range, by an excitation filter unit; receiving the optical signal transmitted in a first wavelength range and the optical signal transmitted in a second wavelength range on the basis of a first directionality of an optical fiber, and outputting a concatenation signal concatenating the transferred optical signals or segmentation signals segmenting a concatenation signal, by a first segmentation/concatenation unit; transferring any one signal among the concatenation signal and the segmentation signal to at least one brain signal measurement target on the basis of the first directionality of the optical fiber, and transferring a measurement signal returning from the at least one brain signal measurement target on the basis of a second directionality of the optical fiber, by a second segmentation/concatenation unit; transmitting the transferred measurement signal in at least one wavelength range among a third wavelength range and a fourth wavelength range, by an emission filter unit; and measuring at least one brain signal of the at least one brain signal measurement target on the basis of the measurement signal transmitted in at least one wavelength range, by a measurement unit.

The first directionality of the optical fiber may represent a directionality of transferring an optical signal from the light source unit to the excitation filter unit, transferring the optical signal from the excitation filter unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality of the optical fiber may represent a directionality of transferring an optical signal returning from the at least one brain signal measurement target to the second segmentation/concatenation unit, transferring the optical signal from the second segmentation/concatenation unit to the emission filter unit, and transferring the optical signal from the emission filter unit to the measurement unit.

According to another aspect of the present invention, there is provided an optical brain signal measurement method comprising the steps of: transferring an optical signal of a first wavelength and an optical signal of a second wavelength through a first optical fiber that transfers the optical signal of a first wavelength transmitted in a first wavelength range and the optical signal of a second wavelength transmitted in a second wavelength range in the first directionality, by a light source unit; outputting a concatenation signal concatenating the optical signal transmitted in a first wavelength range and the optical signal transmitted in a second wavelength range or segmentation signals segmenting a concatenation signal through the first optical fiber, by a first segmentation/concatenation unit; transferring any one signal among the concatenation signal and the segmentation signal to at least one brain signal measurement target through the first optical fiber, and transferring a measurement signal returning from the at least one brain signal measurement target through a second optical fiber that transmits the measurement signal in at least one wavelength range among a third wavelength range and a fourth wavelength range and transfers in the second directionality, by a second segmentation/concatenation unit; and measuring at least one brain signal of the at least one brain signal measurement target on the basis of the transferred measurement signal, by a measurement unit.

The first directionality may represent a directionality of transferring an optical signal from the light source unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality may represent a directionality of transferring an optical signal returning from the at least one brain signal measurement target to the second segmentation/concatenation unit, and transferring the optical signal from the second segmentation/concatenation unit to the measurement unit.

### Advantageous Effects

The present invention may acquire multiple brain signals by using a light source directionality and a light source classification function of an optical fiber and a spectrometer without a dichroic filter.

The present invention may provide an optical fiber photometry technique for acquiring multiple brain signals by utilizing an optical fiber having a specific directionality and a filter function without various lenses and filters.

The present invention may provide an optical brain signal measurement device and method capable of simultaneous recording of light sources of multiple brain signals without configuration of various filters although intuitive and monotonous.

### BRIEF DESCRIPTION OF THE DRAWINGS

FIGS. 1 to 8 are views describing an optical brain signal measurement device according to an embodiment of the present invention.

FIGS. 9 and 10 are views describing an optical brain signal measurement method according to an embodiment of the present invention.

### BEST MODE FOR CARRYING OUT THE INVENTION

An optical brain signal measurement device comprising:
a light source unit for inputting an optical signal of a first wavelength and an optical signal of a second wavelength;
an excitation filter unit for transmitting the optical signal of a first wavelength in a first wavelength range, and transmitting the optical signal of a second wavelength in a second wavelength range;
a first segmentation/concatenation unit for receiving the optical signal transmitted in a first wavelength range and the optical signal transmitted in a second wavelength range on the basis of a first directionality of an optical fiber, and outputting a concatenation signal concatenating the transferred optical signals or segmentation signals segmenting a concatenation signal;
a second segmentation/concatenation unit for transferring any one signal among the concatenation signal and the segmentation signal to at least one brain signal measurement target on the basis of the first directionality of the optical fiber, and transferring a measurement signal returning from the at least one brain signal measurement target on the basis of a second directionality of the optical fiber;
an emission filter unit for transmitting the transferred measurement signal in at least one wavelength range among a third wavelength range and a fourth wavelength range; and
a measurement unit for measuring at least one brain signal of the at least one brain signal measurement target on the basis of the measurement signal transmitted in at least one wavelength range.

Specific structural or functional descriptions of the embodiments according to the concept of the present invention disclosed in this specification are exemplified only for the purpose of explaining the embodiments according to the concept of the present invention, and the embodiments according to the concept of the present invention can be implemented in various forms and are not limited to the embodiments described in this specification.

Since the embodiments according to the concept of the present invention may have various changes and forms, the embodiments will be illustrated in the drawings and described in this specification in detail. However, this is not intended to limit the embodiments according to the concept of the present invention to specific disclosed forms, but includes changes, equivalents, or substitutes included in the spirit and technical scope of the present invention.

Although terms such as first, second, and the like may be used to describe various components, the components should not be limited by the terms. The terms are only intended to distinguish one component from another, for example, a first component may be referred to as a second component, and similarly, a second component may also be referred to as a first component, without departing from the scope according to the concept of the present invention.

When it is mentioned that a certain component is “connected” or “coupled” to another component, it should be understood that although it may be directly connected or coupled to that another component, other components may exist in between. On the other hand, when it is mentioned that a certain component is “directly connected” or “directly connected” to another component, it should be understood that there are no other components in between. Expressions that describe the relationship between the components, such as “between”, “right between”, “directly adjacent to”, and the like, should be interpreted in the same manner.

The terms used in this specification are used only to describe particular embodiments and are not intended to limit the present invention. Singular expressions include plural expressions unless the context clearly dictates otherwise. It should be understood that in this specification, terms such as “comprise”, “have”, and the like intend to indicate the presence of described features, numbers, stages, operations, components, parts, or combinations thereof, and not to exclude in advance the presence or addition of one or more other features, numbers, stages, operations, components, parts, or combinations thereof.

Unless defined otherwise, all terms used herein, including technical or scientific terms, have the same meaning as being commonly understood by those skilled in the art. Terms defined in commonly used dictionaries should be interpreted to have a meaning consistent with the meaning in the context of related technologies, and should not be interpreted in an ideal or excessively formal sense unless explicitly defined in this specification.

Hereinafter, embodiments will be described in detail with reference to the accompanying drawings. However, the scope of the patent application is not limited or restricted by these embodiments. The same reference numerals presented in each drawing represent the same components.

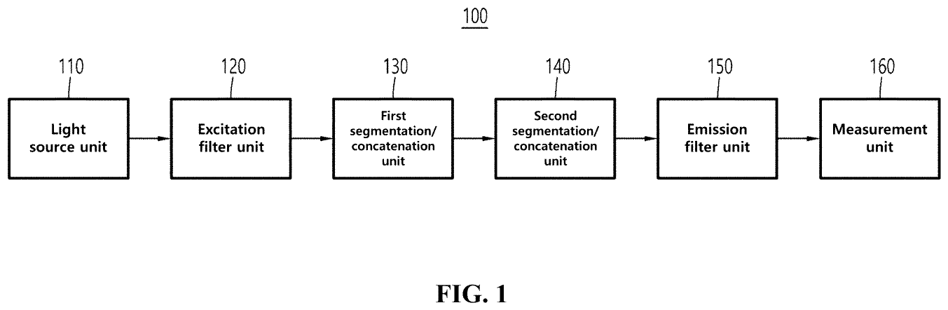

FIGS. 1 to 8 are views describing an optical brain signal measurement device according to an embodiment of the present invention.

FIG. 1 is a view showing components of an optical brain signal measurement device that acquires multiple brain signals (multiple light source signals) using the light source directionality of an optical fiber and a spectrometer without a dichroic filter according to an embodiment of the present invention.

Referring to FIG. 1, an optical brain signal measurement device 100 according to an embodiment of the present invention may include a light source unit 110, an excitation filter unit 120, a first segmentation/concatenation unit 130, a second segmentation/concatenation unit 140, an emission filter unit 150, and a measurement unit 160.

The light source unit 110 according to an embodiment of the present invention inputs an optical signal of a first wavelength and an optical signal of a second wavelength.

For example, the first wavelength may be a wavelength of 405 nm or 465 nm, and the second wavelength may be a wavelength of 473 nm or 560 nm.

For example, the light source unit 110 may include a first light source that inputs an optical signal of a first wavelength and a second light source that inputs an optical signal of a second wavelength.

According to an embodiment of the present invention, the excitation filter unit 120 transmits the optical signal of a first wavelength in a first wavelength range, and transmits the optical signal of a second wavelength in a second wavelength range.

The first wavelength range may be between 400 nm and 410 nm or between 445 nm and 475 nm, and the second wavelength range may be between 465 nm and 495 nm or between 555 nm and 565 nm.

For example, the excitation filter unit 120 may include an excitation filter for transmitting an optical signal of a first wavelength in a first wavelength range and an excitation filter for transmitting an optical signal of a second wavelength in a second wavelength range.

According to an embodiment of the present invention, the first segmentation/concatenation unit 130 may receive an optical signal transmitted in a first wavelength range and an optical signal transmitted in a second wavelength range on the basis of a first directionality of the optical fiber, and output a concatenation signal concatenating the transferred optical signals or segmentation signals segmenting a concatenation signal.

For example, the first directionality of the optical fiber may represent the directionality of transferring an optical signal from a light source unit to the excitation filter unit 120, transferring the optical signal from the excitation filter unit 120 to the first segmentation/concatenation unit 130, transferring the optical signal from the first segmentation/concatenation unit 130 to the second segmentation/concatenation unit 140, and transferring the optical signal from the second segmentation/concatenation unit 140 to the at least one brain signal measurement target.

According to an embodiment of the present invention, the first segmentation/concatenation unit 130 may output a concatenation signal concatenating the transferred optical signals when the at least one brain signal measurement target is one brain signal measurement target, and may output segmentation signals segmenting a concatenation signal when the at least one brain signal measurement target is a plurality of brain signal measurement targets.

For example, in relation to the segmentation signals, the first segmentation/concatenation unit 130 may adjust the segmentation ratio to any one segmentation ratio among 1:10 to 10:1.

For example, when there are two lines for segmenting a concatenation signal and outputting segmented signals, the first segmentation/concatenation unit 130 adjusts the output ratio of optical signals transferred to each line to any one segmentation ratio among 1:10 to 10:1.

That is, the first segmentation/concatenation unit 130 may increase the maximum output signal by adjusting the output ratio of optical signals to 25:75, 10:90, 50:50, or the like.

The numerical range related to the output ratio of optical signals is not limited to the range described above and may be adjusted in various ways within a range that may increase the maximum output signal.

According to an embodiment of the present invention, the second segmentation/concatenation unit 140 may transfer any one signal among the concatenation signal and the segmentation signal to at least one brain signal measurement target on the basis of the first directionality of the optical fiber, and transfer a measurement signal returning from the at least one brain signal measurement target on the basis of the second directionality of the optical fiber.

For example, the second segmentation/concatenation unit 140 may output a concatenation signal concatenating the transferred optical signals in the same manner as the first segmentation/concatenation unit 130 or output segmentation signals segmenting a concatenation signal.

For example, the second directionality of the optical fiber may represent the directionality of transferring an optical signal returning from the at least one brain signal measurement target to the second segmentation/concatenation unit 140, transferring the optical signal from the second segmentation/concatenation unit 140 to the emission filter unit 150, and transferring the optical signal from the emission filter unit 150 to the measurement unit 160.

For example, when the at least one brain signal measurement target is a plurality of brain signal measurement targets, the second segmentation/concatenation unit 140, the emission filter unit 150, and the measurement unit 160 may be additionally configured as many as the number of the plurality of brain signal measurement targets.

According to an embodiment of the present invention, the emission filter unit 150 may transmit the transferred measurement signal in at least one wavelength range among a third wavelength range and a fourth wavelength range.

For example, the third wavelength range may be between 505 nm and 545 nm, and the fourth wavelength range may be between 575 nm and 710 nm.

According to an embodiment of the present invention, the measurement unit 160 may measure at least one brain signal of the at least one brain signal measurement target on the basis of the measurement signal transmitted in at least one wavelength range.

For example, as the measurement unit 160, a spectrometer may be used when the measurement signal transmitted in at least one wavelength range is in a plurality of wavelength ranges, and a photodetector may be used when the measurement signal transmitted in at least one wavelength range is in one wavelength range.

According to an embodiment of the present invention, as the optical brain signal measurement device 100 transfers optical signals to a brain signal measurement target through an optical fiber having directionality, segments or concatenates the signals through a segmentation/concatenation unit, and identifies a wavelength range corresponding to each of multiple signals through a spectrometer, it may acquire multiple brain signals (multiple light source signals) by using the light source directionality of the optical fiber and the spectrometer without a dichroic filter.

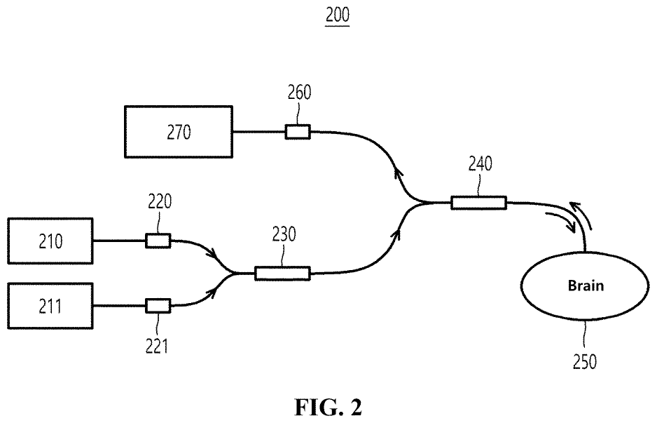

FIG. 2 is a view showing a connection circuit of an optical brain signal measurement device that acquires multiple brain signals (multiple light source signals) using the light source directionality of an optical fiber and a spectrometer without a dichroic filter according to an embodiment of the present invention.

Referring to FIG. 2, according to an embodiment of the present invention, an optical brain signal measurement device 200 may be configured of a first light source 210, a second light source 211, a first excitation filter 220, a second excitation filter 221, a first segmentation/concatenation unit 230, a second segmentation/concatenation unit 240, an emission filter 260, and a measurement unit 270.

The light sources input from the first light source 210 and the second light source 211 transfer an optical signal on the basis of the first directionality of the optical fiber connecting each component to the first segmentation/concatenation unit 230 through the first excitation filter 220 and the second excitation filter 221.

The first wavelength of the light source input by the first light source 210 may be 405 nm or 465 nm, and the second wavelength of the light source input by the second light source 211 may be 473 nm or 560 nm.

The first excitation filter 220 transmits signals in a first wavelength range, and the first wavelength range may be between 400 nm and 410 nm or between 445 nm and 475 nm.

The second excitation filter 221 transmits signals in a second wavelength range, and the second wavelength range may be between 465 nm and 495 nm or between 555 nm and 565 nm.

The first segmentation/concatenation unit 230 transfers a concatenation signal to the second segmentation/concatenation unit 240 on the basis of the first directionality.

The second segmentation/concatenation unit 240 transfers the optical signal to a brain signal measurement target 250 on the basis of the first directionality, and receives a measurement signal returning from the brain signal measurement target 250 on the basis of the second directionality and transfers the received measurement signal to the emission filter 260.

The emission filter 260 transmits the transferred measurement signal in at least one wavelength range among a third wavelength range and a fourth wavelength range.

For example, the third wavelength range may be between 505 nm and 545 nm, and the fourth wavelength range may be between 575 nm and 710 nm.

The measurement unit 270 may be a spectrometer when the measurement signal transmitted in at least one wavelength range is in a plurality of wavelength ranges, and a photodetector when the measurement signal transmitted in at least one wavelength range is in one wavelength range.

The measurement unit 270 detects the signal using the photodetector when the measurement signal is a brain signal in one wavelength range, and detects the signal using the spectrometer when the measurement signal is two or more brain signals in a plurality of wavelength ranges.

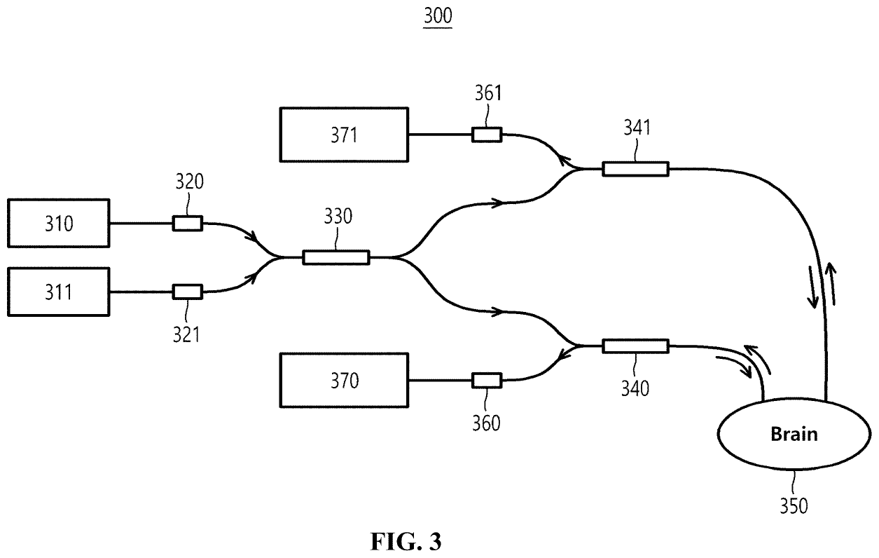

FIG. 3 is a view showing a connection circuit of an optical brain signal measurement device that acquires multiple brain signals (multiple light source signals) from a plurality of parts of a brain signal measurement target using the light source directionality of an optical fiber and a spectrometer without a dichroic filter according to an embodiment of the present invention.

Referring to FIG. 3, according to an embodiment of the present invention, an optical brain signal measurement device 300 may be configured of a first light source 310, a second light source 311, a first excitation filter 320, a second excitation filter 321, a first segmentation/concatenation unit 330, a second segmentation/concatenation unit 340, a second segmentation/concatenation unit 341, an emission filter 360, an emission filter 361, a measurement unit 370, and a measurement unit 371.

The light sources input from the first light source 310 and the second light source 311 transfer an optical signal on the basis of the first directionality of the optical fiber connecting each component to the first segmentation/concatenation unit 330 through the first excitation filter 320 and the second excitation filter 321.

The first wavelength of the light source input by the first light source 310 may be 405 nm or 465 nm, and the second wavelength of the light source input by the second light source 311 may be 473 nm or 560 nm.

The first excitation filter 320 transmits signals in a first wavelength range, and the first wavelength range may be between 400 nm and 410 nm or between 445 nm and 475 nm.

The second excitation filter 321 transmits signals in a second wavelength range, and the second wavelength range may be between 465 nm and 495 nm or between 555 nm and 565 nm.

The first segmentation/concatenation unit 330 transfers segmentation signals to the second segmentation/concatenation unit 340 and the second segmentation/concatenation unit 341 on the basis of the first directionality. For example, the segmentation signals may be signals segmented at the same ratio, and the segmentation ratio may be determined on the basis of user's setting.

For example, in relation to the segmentation signals, the first segmentation/concatenation unit 330 may adjust the segmentation ratio to any one segmentation ratio among 1:10 to 10:1.

For example, when there are two lines for segmenting a concatenation signal and outputting segmented signals, the first segmentation/concatenation unit 330 adjusts the output ratio of optical signals transferred to each line to any one segmentation ratio among 1:10 to 10:1.

That is, the first segmentation/concatenation unit 330 may increase the maximum output signal by adjusting the output ratio of optical signals to 25:75, 10:90, 50:50, or the like.

The second segmentation/concatenation unit 340 and the second segmentation/concatenation unit 341 transfer the optical signals to a plurality of parts of a brain signal measurement target 350 on the basis of the first directionality, and receive measurement signals returning from the brain signal measurement target 350 on the basis of the second directionality and transfer the received measurement signals to the emission filter 360 and the emission filter 361.

The emission filter 360 and the emission filter 361 transmit the transferred measurement signals in at least one wavelength range among a third wavelength range and a fourth wavelength range.

For example, the third wavelength range may be between 505 nm and 545 nm, and the fourth wavelength range may be between 575 nm and 710 nm.

As the measurement unit 370 and the measurement unit 371, a spectrometer may be used when the measurement signal transmitted in at least one wavelength range is in a plurality of wavelength ranges, and a photodetector may be used when the measurement signal transmitted in at least one wavelength range is in one wavelength range.

The measurement unit 370 and the measurement unit 371 may detect the signal using the photodetector when the measurement signal is a brain signal in one wavelength range, and detect the signal using the spectrometer when the measurement signal is two or more brain signals in a plurality of wavelength ranges.

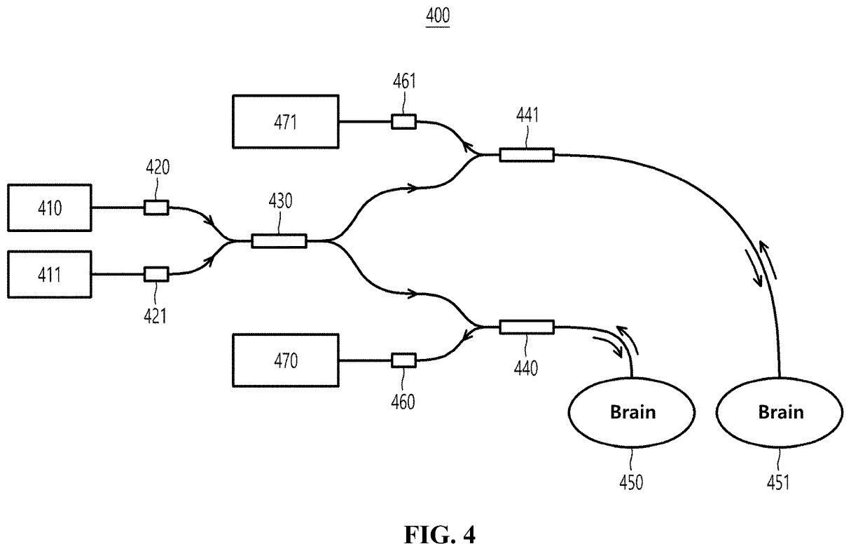

FIG. 4 is a view showing a connection circuit of an optical brain signal measurement device that acquires multiple brain signals (multiple light source signals) from a plurality of brain signal measurement targets using the light source directionality of an optical fiber and a spectrometer without a dichroic filter according to an embodiment of the present invention.

Referring to FIG. 4, according to an embodiment of the present invention, an optical brain signal measurement device 400 may be configured of a first light source 410, a second light source 411, a first excitation filter 420, a second excitation filter 421, a first segmentation/concatenation unit 430, a second segmentation/concatenation unit 440, a second segmentation/concatenation unit 441, an emission filter 460, an emission filter 461, a measurement unit 470, and a measurement unit 471.

According to an embodiment of the present invention, the optical brain signal measurement device 400 operates in the same manner as the optical brain signal measurement device 300 described in FIG. 3, and may measure brain signals from a brain signal measurement target 450 and a brain signal measurement target 451.

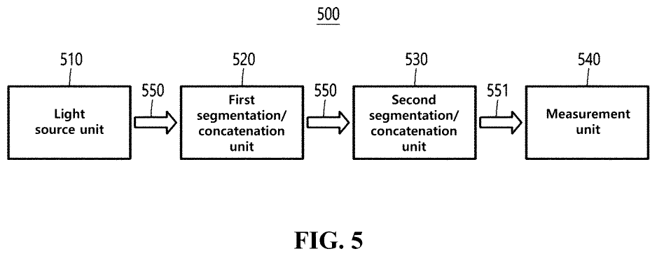

FIG. 5 is a view showing components of an optical brain signal measurement device that acquires multiple brain signals using an optical fiber having a light source classification function without a dichroic filter, an excitation filter, or an emission filter according to an embodiment of the present invention.

Referring to FIG. 5, an optical brain signal measurement device 500 according to an embodiment of the present invention may include a light source unit 510, a first segmentation/concatenation unit 520, a second segmentation/concatenation unit 530, and a measurement unit 540, and the light source unit 510, the first segmentation/concatenation unit 520, and the second segmentation/concatenation unit 530 may be connected through a first optical fiber 550, and the second segmentation/concatenation unit 530 and the measurement unit 540 may be connected through a second optical fiber 551.

According to an embodiment of the present invention, the light source unit 510 may transfer an optical signal of a first wavelength and an optical signal of a second wavelength through a first optical fiber 550 that transfers an optical signal of a first wavelength transmitted in a first wavelength range and an optical signal of a second wavelength transmitted in a second wavelength range in the first directionality.

For example, the first segmentation/concatenation unit 520 outputs a concatenation signal concatenating an optical signal transmitted in a first wavelength range and an optical signal transmitted in a second wavelength range or segmentation signals segmenting a concatenation signal through the first optical fiber 550.

For example, in relation to the segmentation signals, the first segmentation/concatenation unit 520 may adjust the segmentation ratio to any one segmentation ratio among 1:10 to 10:1.

For example, when there are two lines for segmenting a concatenation signal and outputting segmented signals, the first segmentation/concatenation unit 520 adjusts the output ratio of optical signals transferred to each line to any one segmentation ratio among 1:10 to 10:1.

That is, the first segmentation/concatenation unit 520 may increase the maximum output signal by adjusting the output ratio of optical signals to 25:75, 10:90, 50:50, or the like.

The numerical range related to the output ratio of optical signals is not limited to the range described above and may be adjusted in various ways within a range that may increase the maximum output signal.

According to an embodiment of the present invention, the second segmentation/concatenation unit 530 transfers any one signal among the concatenation signal and the segmentation signal to at least one brain signal measurement target through the first optical fiber 550.

For example, the second segmentation/concatenation unit 140 may output a concatenation signal concatenating the transferred optical signals in the same manner as the first segmentation/concatenation unit 130 or output segmentation signals segmenting a concatenation signal.

In addition, the second segmentation/concatenation unit 530 may transfer a measurement signal returning from at least one brain signal measurement target through the second optical fiber 551 that transmits the measurement signal in at least one wavelength range among a third wavelength range and a fourth wavelength range and transfers in the second directionality.

According to an embodiment of the present invention, the measurement unit 540 may measure at least one brain signal of at least one brain signal measurement target on the basis of the transferred measurement signal.

According to an embodiment of the present invention, the first optical fiber 550 classifies and transmits the wavelength of a light source so that the wavelength range of a concatenation signal concatenated in the first segmentation/concatenation unit 520 is between 473 nm and 561 nm when the wavelength input from the first light source is 465 nm and the wavelength input from the second light source is 560 nm.

According to an embodiment of the present invention, the second optical fiber 551 classifies and transmits the wavelength of a light source so that the wavelength range of the measurement signal transferred to the measurement unit 540 is between 532 nm and 670 nm.

According to an embodiment of the present invention, as the optical brain signal measurement device 500 transfers optical signals to a brain signal measurement target through an optical fiber having directionality and a wavelength transmission (classification) function, segments or concatenates the signals through a segmentation/concatenation unit, and identifies a wavelength range corresponding to each of multiple signals through a spectrometer, it may acquire multiple brain signals (multiple light source signals) without a dichroic filter, an excitation filter, and an emission filter.

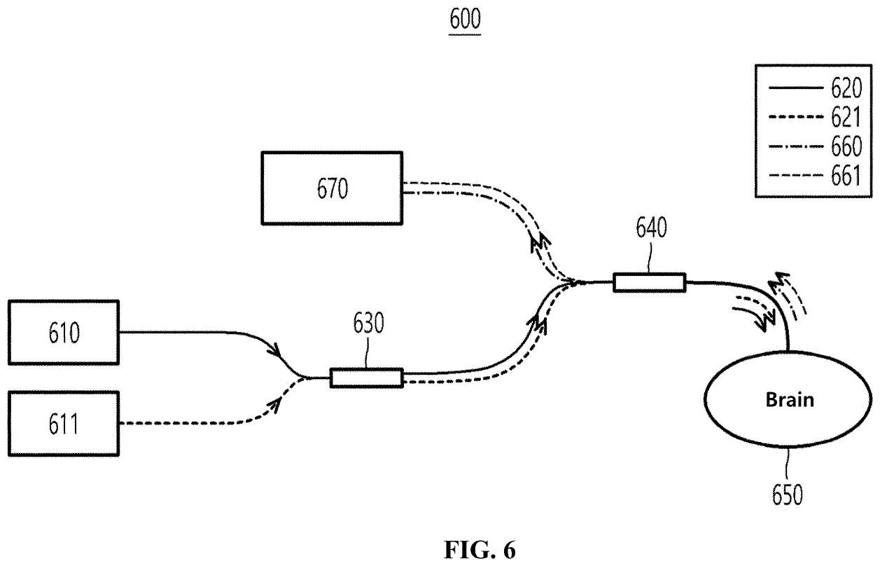

FIG. 6 is a view showing a connection circuit of an optical brain signal measurement device that acquires multiple brain signals using an optical fiber having a light source classification function without a dichroic filter, an excitation filter, or an emission filter according to an embodiment of the present invention.

Referring to FIG. 6, according to an embodiment of the present invention, an optical brain signal measurement device 600 may be configured of a first light source 610, a second light source 611, a first segmentation/concatenation unit 630, a second segmentation/concatenation unit 640, and a measurement unit 670.

An optical signal 620 of the first light source 610 passing through the first optical fiber and transmitted through a wavelength range and an optical signal 621 of the second light source 611 passing through the first optical fiber and transmitted through a wavelength range are transferred to the first segmentation/concatenation unit 630, and the optical signal 620 and the optical signal 621 concatenated in the first segmentation/concatenation unit 630 are transferred to the second segmentation/concatenation unit 640.

The second segmentation/concatenation unit 640 transfers the optical signal 620 and the optical signal 621 to a brain signal measurement target 650, and transfers an optical signal 660 and an optical signal 661 passing through the second optical fiber and transmitted through a wavelength range to the measurement unit 670.

According to an embodiment of the present invention, the optical brain signal measurement device 600 is an optical fiber photometry technique without a dichroic filter, an excitation filter, and an emission filter by using directionality of a light source and a multi-functional optical fiber that transfers only light sources of a specific wavelength, and may record multiple brain signal light sources.

According to an embodiment of the present invention, the first optical fiber related to the optical signal 620 and the optical signal 621 classifies and transmits the wavelength of a light source so that the wavelength range of a concatenation signal concatenated in the first segmentation/concatenation unit 630 is between 473 nm and 561 nm when the wavelength input from the first light source is 465 nm and the wavelength input from the second light source is 560 nm.

According to an embodiment of the present invention, the second optical fiber related to the optical signal 660 and the optical signal 661 classifies and transmits the wavelength of a light source so that the wavelength range of the measurement signal transferred to the measurement unit 670 is between 532 nm and 670 nm.

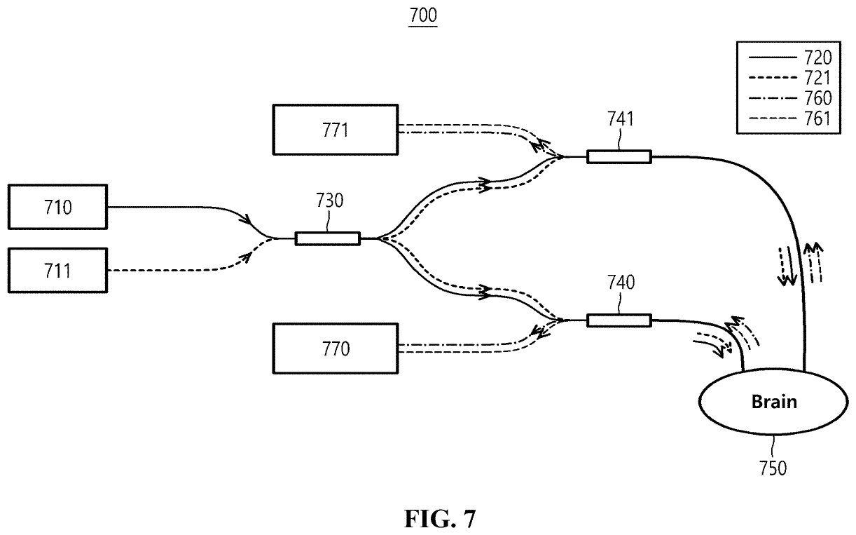

FIG. 7 is a view showing a connection circuit of an optical brain signal measurement device that acquires multiple brain signals for a plurality of parts of a brain signal measurement target using an optical fiber having a light source classification function without a dichroic filter, an excitation filter, or an emission filter according to an embodiment of the present invention.

Referring to FIG. 7, according to an embodiment of the present invention, an optical brain signal measurement device 700 may be configured of a first light source 710, a second light source 711, a first segmentation/concatenation unit 730, a second segmentation/concatenation unit 740, a second segmentation/concatenation unit 741, and a measurement unit 770, and a measurement unit 771.

An optical signal 720 of the first light source 710 passing through the first optical fiber and transmitted through a wavelength range and an optical signal 721 of the second light source 711 passing through the first optical fiber and transmitted through a wavelength range are transferred to the first segmentation/concatenation unit 730, and the optical signal 720 and the optical signal 721 segmented in the first segmentation/concatenation unit 730 are transferred to the second segmentation/concatenation unit 740 and the second segmentation/concatenation unit 741.

For example, in relation to the segmentation signals, the first segmentation/concatenation unit 730 may adjust the segmentation ratio to any one segmentation ratio among 1:10 to 10:1.

For example, when there are two lines for segmenting a concatenation signal and outputting segmented signals, the first segmentation/concatenation unit 730 adjusts the output ratio of optical signals transferred to each line to any one segmentation ratio among 1:10 to 10:1.

That is, the first segmentation/concatenation unit 730 may increase the maximum output signal by adjusting the output ratio of optical signals to 25:75, 10:90, 50:50, or the like.

The numerical range related to the output ratio of optical signals is not limited to the range described above and may be adjusted in various ways within a range that may increase the maximum output signal.

The second segmentation/concatenation unit 740 transfers the optical signal 720 and the optical signal 721 to a brain signal measurement target 750, and transfers an optical signal 760 and an optical signal 761 passing through the second optical fiber and transmitted through a wavelength range to the measurement unit 770 and the measurement unit 771.

For example, the second segmentation/concatenation unit 740 may output a concatenation signal concatenating the transferred optical signals in the same manner as the first segmentation/concatenation unit 730 or output segmentation signals segmenting a concatenation signal.

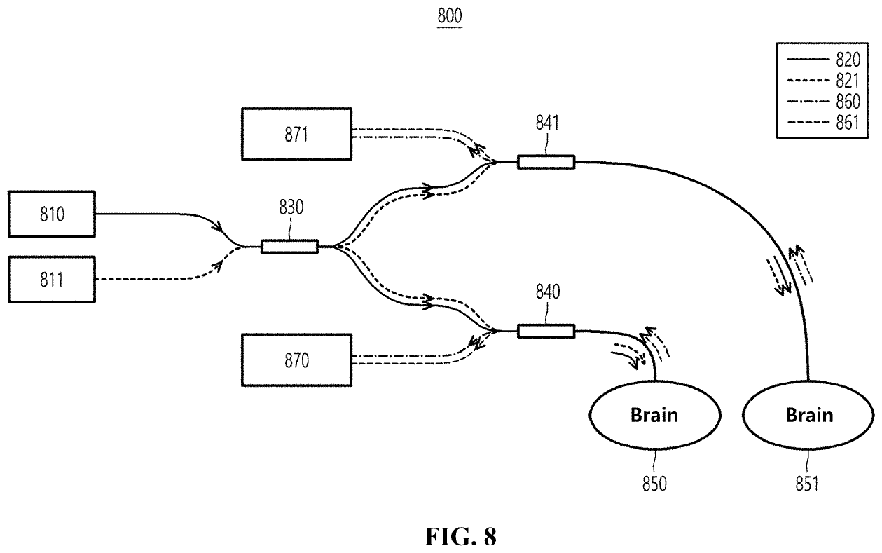

FIG. 8 is a view showing a connection circuit of an optical brain signal measurement device that acquires multiple brain signals for a plurality of brain signal measurement targets using an optical fiber having a light source classification function without a dichroic filter, an excitation filter, or an emission filter according to an embodiment of the present invention.

Referring to FIG. 8, according to an embodiment of the present invention, an optical brain signal measurement device 800 may be configured of a first light source 810, a second light source 811, a first segmentation/concatenation unit 830, a second segmentation/concatenation unit 840, a second segmentation/concatenation unit 841, a measurement unit 870, and a measurement unit 871.

An optical signal 820 of the first light source 810 passing through the first optical fiber and transmitted through a wavelength range and an optical signal 821 of the second light source 811 passing through the first optical fiber and transmitted through a wavelength range are transferred to the first segmentation/concatenation unit 830, and the optical signal 820 and the optical signal 821 segmented in the first segmentation/concatenation unit 830 are transferred to the second segmentation/concatenation unit 840 and the second segmentation/concatenation unit 841.

The second segmentation/concatenation unit 840 transfers the optical signal 820 and the optical signal 821 to a brain signal measurement target 850 and a brain signal measurement target 851, and transfers an optical signal 860 and an optical signal 861 passing through the second optical fiber and transmitted through a wavelength range to the measurement unit 870 and the measurement unit 871.

According to an embodiment of the present invention, a brain signal measurement device may measure brain signals by measuring the optical fiber luminous intensity of multiple brain signal recordings using an optical fiber having a light source classification function without an excitation filter and an emission filter, as well as a dichroic filter.

Therefore, the present invention may provide an optical brain signal measurement device and method capable of simultaneous recording of light sources of multiple brain signals without configuration of various filters although intuitive and monotonous.

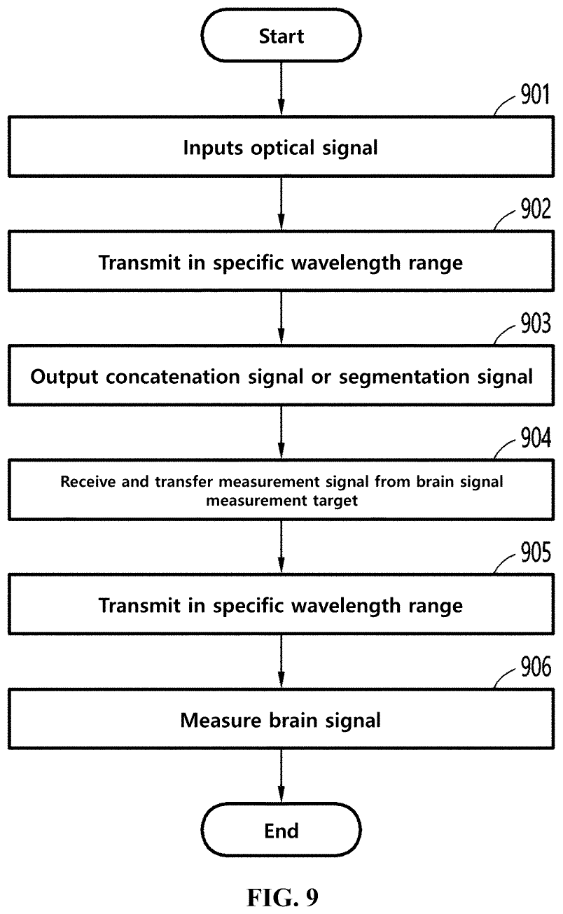

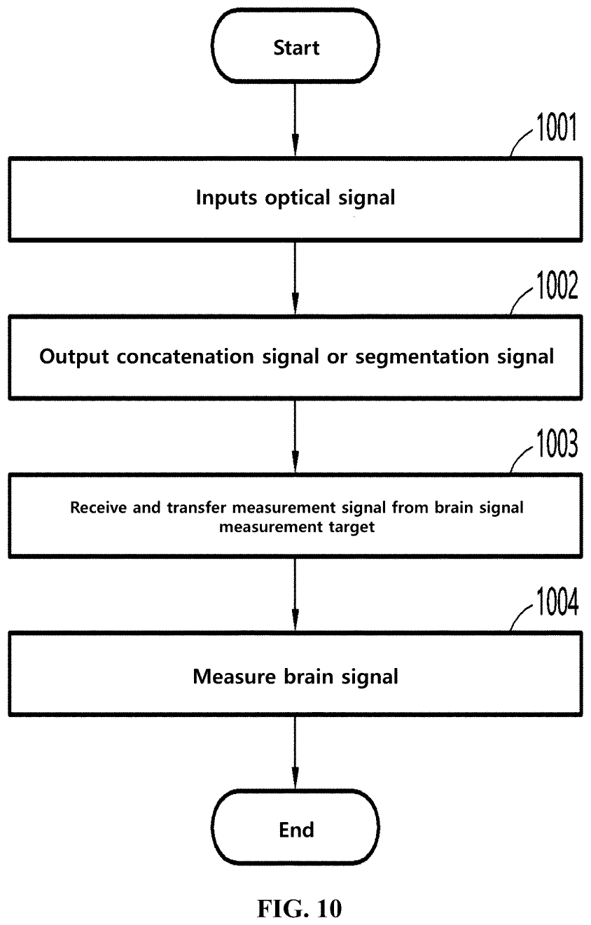

FIGS. 9 and 10 are views describing an optical brain signal measurement method according to an embodiment of the present invention.

FIG. 9 is a flowchart illustrating an optical brain signal measurement method of acquiring multiple brain signals (multiple light source signals) using the light source directionality of an optical fiber and a spectrometer without a dichroic filter according to an embodiment of the present invention.

Referring to FIG. 9, at step 901, the optical brain signal measurement method according to an embodiment of the present invention inputs an optical signal.

That is, the optical brain signal measurement method according to an embodiment of the present invention may input an optical signal of a first wavelength and an optical signal of a second wavelength.

At step 902, the optical brain signal measurement method according to an embodiment of the present invention transmits an optical signal in a specific wavelength range.

That is, the optical brain signal measurement method according to an embodiment of the present invention may transmit an optical signal of a first wavelength in a first wavelength range, and transmit an optical signal of a second wavelength in a second wavelength range.

At step 903, the optical brain signal measurement method according to an embodiment of the present invention outputs a concatenation signal or a segmentation signal.

That is, the optical brain signal measurement method according to an embodiment of the present invention may receive an optical signal transmitted in a first wavelength range and an optical signal transmitted in a second wavelength range on the basis of a first directionality of the optical fiber, and output a concatenation signal concatenating the transferred optical signals or segmentation signals segmenting a concatenation signal.

At step 904, the optical brain signal measurement method according to an embodiment of the present invention receives and transfers a measurement signal from a brain signal measurement target.

That is, the optical brain signal measurement method according to an embodiment of the present invention may transfer any one signal among the concatenation signal and the segmentation signal to at least one brain signal measurement target on the basis of the first directionality of the optical fiber, and transfer a measurement signal returning from at least one brain signal measurement target on the basis of the second directionality of the optical fiber.

At step 905, the optical brain signal measurement method according to an embodiment of the present invention transmits an optical signal in a specific wavelength range.

That is, the optical brain signal measurement method according to an embodiment of the present invention may transmit the transferred measurement signal in at least one wavelength range among a third wavelength range and a fourth wavelength range.

At step 906, the optical brain signal measurement method according to an embodiment of the present invention measures brain signals.

That is, the optical brain signal measurement method according to an embodiment of the present invention may measure at least one brain signal of at least one brain signal measurement target on the basis of the measurement signal transmitted in at least one wavelength range.

FIG. 10 is a flowchart illustrating an optical brain signal measurement method of acquiring multiple brain signals using an optical fiber having a light source classification function without a dichroic filter, an excitation filter, or an emission filter according to an embodiment of the present invention.

Referring to FIG. 10, at step 1001, the optical brain signal measurement method according to an embodiment of the present invention inputs an optical signal.

That is, the optical brain signal measurement method according to an embodiment of the present invention may transfer an optical signal of a first wavelength and an optical signal of a second wavelength through a first optical fiber that transfers an optical signal of a first wavelength transmitted in a first wavelength range and an optical signal of a second wavelength transmitted in a second wavelength range in the first directionality.

At step 1002, the optical brain signal measurement method according to an embodiment of the present invention outputs a concatenation signal or a segmentation signal.

That is, the optical brain signal measurement method according to an embodiment of the present invention may output a concatenation signal concatenating an optical signal transmitted in a first wavelength range and an optical signal transmitted in a second wavelength range or segmentation signals segmenting a concatenation signal through the first optical fiber.

At step 1003, the optical brain signal measurement method according to an embodiment of the present invention receives and transfers a measurement signal from a brain signal measurement target.

That is, the optical brain signal measurement method according to an embodiment of the present invention may transfer any one signal among the concatenation signal and the segmentation signal to at least one brain signal measurement target through the first optical fiber, and transfer a measurement signal returning from at least one brain signal measurement target through a second optical fiber that transmits the measurement signal in at least one wavelength range among a third wavelength range and a fourth wavelength range and transfers in the second directionality.

At step 1004, the optical brain signal measurement method according to an embodiment of the present invention measures brain signals.

That is, the optical brain signal measurement method according to an embodiment of the present invention may measure at least one brain signal of at least one brain signal measurement target on the basis of the measurement signal transmitted in at least one wavelength range.

Therefore, the present invention may provide an optical brain signal measurement device and method capable of simultaneous recording of light sources of multiple brain signals without configuration of various filters although intuitive and monotonous.

The device described above may be implemented as hardware components, software components, and/or combinations of the hardware components and the software components. For example, the device and components described in the embodiments may be implemented using one or more general-purpose computers or special-purpose computers, such as, for example, a processor, a controller, an arithmetic logic unit (ALU), a digital signal processor, a microcomputer, a field programmable array (FPA), a programmable logic unit (PLU), a microprocessor, or any other devices capable of executing instructions and responding thereto. The processing device may execute an operating system (OS) and one or more software applications executed on the operating system. In addition, the processing device may access, store, manipulate, process, and generate data in response to the execution of software. Although there are cases where it is described that one processing device is used for convenience of understanding, those skilled in the art will appreciate that the processing device may include a plurality of processing elements and/or several types of processing elements. For example, the processing unit may include a plurality of processors, or a processor and a controller. In addition, other processing configurations, such as parallel processors, are also possible.

The method according to an embodiment may be implemented in the form of program instructions that can be executed through various computer means and recorded on a computer-readable medium. The computer-readable medium may include program instructions, data files, data structures, and the like alone or in combination. The program instructions recorded on the medium may be those specially designed and configured for the embodiment or may be those known to and used by those skilled in the art of computer software. Examples of the computer-readable recording medium include magnetic media such as hard disks, floppy disks, and magnetic tapes, optical media such as CD-ROMs and DVDs, magneto-optical media such as floptical disks, and hardware devices specially configured to store and execute program instructions, such as ROMs, RAMs, flash memories, and the like. Examples of the program instructions include high-level language codes that can be executed by a computer using an interpreter or the like, as well as machine language codes generated by a compiler. The hardware devices described above may be configured to operate as one or more software modules to perform the operations of the embodiment, and vice versa.

The software may include a computer program, code, instructions, or a combination of one or more of these, and the processing device may be configured to perform a desired operation or may command the processing device independently or collectively. The software and/or data may be permanently or temporarily embodied in any type of machine, component, physical device, virtual equipment, computer storage medium or device, or transmitted signal waves to be interpreted by the processing device or to provide instructions or data to the processing device. The software may be distributed to computer systems connected through a network to be stored or executed in a distributed manner. The software and data may be stored on one or more computer-readable recording media.

Although the embodiments have been described through limited drawings as described above, those skilled in the art may make various modifications and variations from the above descriptions. For example, although the described techniques are performed in an order different from that of the described method, and/or components such as the described systems, structures, devices, circuits, and the like are coupled or combined in a form different from those of the described methods or replaced or substituted by other components or equivalents, appropriate results can be achieved.

Therefore, other implementations, other embodiments, and equivalents to the claims also fall within the scope of the claims described below.

### Industrial Applicability

The present invention relates to an optical brain signal measurement device and a method thereof, and more particularly, to an optical fiber photometry technique for acquiring multiple brain signals by using a light source directionality and a light source classification function of an optical fiber and a spectrometer without a dichroic filter.

1. An optical brain signal measurement device comprising:
a light source unit for inputting an optical signal of a first wavelength and an optical signal of a second wavelength;
an excitation filter unit for transmitting the optical signal of a first wavelength in a first wavelength range, and transmitting the optical signal of a second wavelength in a second wavelength range;
a first segmentation/concatenation unit for receiving the optical signal transmitted in a first wavelength range and the optical signal transmitted in a second wavelength range on the basis of a first directionality of an optical fiber, and outputting a concatenation signal concatenating the transferred optical signals or segmentation signals segmenting a concatenation signal;
a second segmentation/concatenation unit for transferring any one signal among the concatenation signal and the segmentation signal to at least one brain signal measurement target on the basis of the first directionality of the optical fiber, and transferring a measurement signal returning from the at least one brain signal measurement target on the basis of a second directionality of the optical fiber;
an emission filter unit for transmitting the transferred measurement signal in at least one wavelength range among a third wavelength range and a fourth wavelength range; and
a measurement unit for measuring at least one brain signal of the at least one brain signal measurement target on the basis of the measurement signal transmitted in at least one wavelength range.

2. The device according to claim 1, wherein the first directionality of the optical fiber represents a directionality of transferring an optical signal from the light source unit to the excitation filter unit, transferring the optical signal from the excitation filter unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality of the optical fiber represents a directionality of transferring an optical signal returning from the at least one brain signal measurement target to the second segmentation/concatenation unit, transferring the optical signal from the second segmentation/concatenation unit to the emission filter unit, and transferring the optical signal from the emission filter unit to the measurement unit.

3. The device according to claim 1, wherein when the at least one brain signal measurement target is a plurality of brain signal measurement targets, the second segmentation/concatenation unit, the emission filter unit, and the measurement unit are additionally configured as many as the number of the plurality of brain signal measurement targets.

4. The device according to claim 1, wherein the first segmentation/concatenation unit outputs a concatenation signal concatenating the transferred optical signals when the at least one brain signal measurement target is one brain signal measurement target, and outputs segmentation signals segmenting a concatenation signal when the at least one brain signal measurement target is a plurality of brain signal measurement targets.

5. The device according to claim 4, wherein in relation to the segmentation signals, the first segmentation/concatenation unit adjusts a segmentation ratio to any one segmentation ratio among 1:10 to 10:1.

6. The device according to claim 1, wherein the measurement unit is a spectrometer when the measurement signal transmitted in at least one wavelength range is in a plurality of wavelength ranges, and a photodetector when the measurement signal transmitted in at least one wavelength range is in one wavelength range.

7. An optical brain signal measurement device comprising:
a light source unit for transferring an optical signal of a first wavelength and an optical signal of a second wavelength through a first optical fiber that transfers the optical signal of a first wavelength transmitted in a first wavelength range and the optical signal of a second wavelength transmitted in a second wavelength range in the first directionality;
a first segmentation/concatenation unit for outputting a concatenation signal concatenating the optical signal transmitted in a first wavelength range and the optical signal transmitted in a second wavelength range or segmentation signals segmenting a concatenation signal through the first optical fiber;
a second segmentation/concatenation unit for transferring any one signal among the concatenation signal and the segmentation signal to at least one brain signal measurement target through the first optical fiber, and transferring a measurement signal returning from the at least one brain signal measurement target through a second optical fiber that transmits the measurement signal in at least one wavelength range among a third wavelength range and a fourth wavelength range and transfers in the second directionality; and
a measurement unit for measuring at least one brain signal of the at least one brain signal measurement target on the basis of the transferred measurement signal.

8. The device according to claim 7, wherein the first directionality represents a directionality of transferring an optical signal from the light source unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality represents a directionality of transferring an optical signal returning from the at least one brain signal measurement target to the second segmentation/concatenation unit, and transferring the optical signal from the second segmentation/concatenation unit to the measurement unit.

9. The device according to claim 7, wherein when the at least one brain signal measurement target is a plurality of brain signal measurement targets, the second segmentation/concatenation unit and the measurement unit are additionally configured as many as the number of the plurality of brain signal measurement targets.

10. The device according to claim 7, wherein the first segmentation/concatenation unit outputs a concatenation signal concatenating the transferred optical signals when the at least one brain signal measurement target is one brain signal measurement target, and outputs segmentation signals segmenting a concatenation signal when the at least one brain signal measurement target is a plurality of brain signal measurement targets.

11. The device according to claim 10, wherein in relation to the segmentation signals, the first segmentation/concatenation unit adjusts a segmentation ratio to any one segmentation ratio among 1:10 to 10:1.

12. The device according to claim 7, wherein the measurement unit is at least one among a spectrometer and a photodetector.

13. An optical brain signal measurement method comprising the steps of:
inputting an optical signal of a first wavelength and an optical signal of a second wavelength, by a light source unit;
transmitting the optical signal of a first wavelength in a first wavelength range, and transmitting the optical signal of a second wavelength in a second wavelength range, by an excitation filter unit;
receiving the optical signal transmitted in a first wavelength range and the optical signal transmitted in a second wavelength range on the basis of a first directionality of an optical fiber, and outputting a concatenation signal concatenating the transferred optical signals or segmentation signals segmenting a concatenation signal, by a first segmentation/concatenation unit;
transferring any one signal among the concatenation signal and the segmentation signal to at least one brain signal measurement target on the basis of the first directionality of the optical fiber, and transferring a measurement signal returning from the at least one brain signal measurement target on the basis of a second directionality of the optical fiber, by a second segmentation/concatenation unit;
transmitting the transferred measurement signal in at least one wavelength range among a third wavelength range and a fourth wavelength range, by an emission filter unit; and
measuring at least one brain signal of the at least one brain signal measurement target on the basis of the measurement signal transmitted in at least one wavelength range, by a measurement unit.

14. The method according to claim 12, wherein the first directionality of the optical fiber represents a directionality of transferring an optical signal from the light source unit to the excitation filter unit, transferring the optical signal from the excitation filter unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality of the optical fiber represents a directionality of transferring an optical signal returning from the at least one brain signal measurement target to the second segmentation/concatenation unit, transferring the optical signal from the second segmentation/concatenation unit to the emission filter unit, and transferring the optical signal from the emission filter unit to the measurement unit.

15. An optical brain signal measurement method comprising the steps of:
transferring an optical signal of a first wavelength and an optical signal of a second wavelength through a first optical fiber that transfers the optical signal of a first wavelength transmitted in a first wavelength range and the optical signal of a second wavelength transmitted in a second wavelength range in the first directionality, by a light source unit;
outputting a concatenation signal concatenating the optical signal transmitted in a first wavelength range and the optical signal transmitted in a second wavelength range or segmentation signals segmenting a concatenation signal through the first optical fiber, by a first segmentation/concatenation unit;
transferring any one signal among the concatenation signal and the segmentation signal to at least one brain signal measurement target through the first optical fiber, and transferring a measurement signal returning from the at least one brain signal measurement target through a second optical fiber that transmits the measurement signal in at least one wavelength range among a third wavelength range and a fourth wavelength range and transfers in the second directionality, by a second segmentation/concatenation unit; and
measuring at least one brain signal of the at least one brain signal measurement target on the basis of the transferred measurement signal, by a measurement unit.

16. The method according to claim 15, wherein the first directionality represents a directionality of transferring an optical signal from the light source unit to the first segmentation/concatenation unit, transferring the optical signal from the first segmentation/concatenation unit to the second segmentation/concatenation unit, and transferring the optical signal from the second segmentation/concatenation unit to the at least one brain signal measurement target, and the second directionality represents a directionality of transferring an optical signal returning from the at least one brain signal measurement target to the second segmentation/concatenation unit, and transferring the optical signal from the second segmentation/concatenation unit to the measurement unit.
